(19) United States
(12) Patent Application Publication
(10) Pub. No.: US 2019/0217128 A1
(43) Pub. Date: Jul. 18, 2019

(54) SYSTEM AND METHOD FOR SONOGENETIC THERAPY

(71) Applicant: Circuit Therapeutics, Inc., Mt. View, CA (US)

(72) Inventors: Brian Andrew Ellingwood, Sunnyvale, CA (US); Brian Beckey, Redwood City, CA (US); Christopher L. Towne, San Francisco, CA (US); Dan Andersen, Menlo Park, CA (US)

(73) Assignee: Circuit Therapeutics, Inc., Mt. View, CA (US)

(21) Appl. No.: 16/249,891

(22) Filed: Jan. 16, 2019

Related U.S. Application Data
(60) Provisional application No. 62/617,921, filed on Jan. 16, 2018.

(51) Int. Cl. A61N 7/00; A61B 17/34

(52) U.S. Cl. CPC A61N 7/00; A61N 2007/0047; A61N 2007/0026; A61B 17/3468

(57) ABSTRACT

One embodiment is directed to an implantable probe system for delivering acoustical energy to a targeted tissue portion of a patient, comprising: a plurality of substrate portions, each substrate portion comprising at least one acoustical emitter; a probe body portion having proximal and distal ends and being movably coupled to the plurality of substrates and configured to at least partially encapsulate the plurality of substrates; and a distal end portion coupled to the distal end of the probe body portion, the distal end portion comprising at least one guiding feature configured to redirect a path of at least one of the substrate portions as such substrate portion is extended through and past the distal end portion by moving the plurality of substrates relative to the probe body portion

### RELATED APPLICATION DATA

The present application claims priority to U.S. Provisional Application Ser. No. 62/617,921, filed Jan. 16, 2018. The foregoing application is hereby incorporated by reference into the present application in its entirety.

### FIELD OF THE INVENTION

The present invention relates to therapeutic intervention using sound to activate mechanosensitive cellular transmembrane proteins (mechanoreceptors) that in turn either excite or inhibit neural function.

### BACKGROUND

Pulsed ultrasound (hereinafter, “US”) at acoustic intensities less than 500 mW/cm² has been shown to activate neurons without producing thermal effects or tissue damage in small animals such as rodents. However, delivering equivalent acoustic dosages to reach meaningful depths in tissue such as the human brain typically requires the use of large transducers and commensurate output powers. Such systems have been described, for example, in U.S. Pat. Nos. 5,526,815 & 8,591,419, which are incorporated by reference herein in their entirety, and which generally are not suitable configurations as implantable or wearable systems. There is a need for systems, methods, techniques, and configurations which may be utilized to specifically and minimally invasively control certain aspects of the neurological system, such as to assist in addressing certain neurological disorders. Disclosed herein are certain systems, methods, techniques, and configurations which may combine the use of certain genetic materials with minimally invasive energy delivery techniques to provide aspects of desired therapeutic paradigms.

Another embodiment is directed to a system for altering the function of a sensory unit that innervates a targeted tissue portion of an animal, the system comprising an acoustical source configured to be operatively coupled to an exposed surface of the animal and to provide acoustical energy to the targeted tissue portion, wherein the sensory unit has been configured to express an acoustically sensitive transmembrane protein, such that when the targeted tissue portion is exposed to acoustical energy transcutaneously from the acoustical source, a membrane potential of cells comprising the targeted tissue structure is modulated at least in part due to exposure of the acoustically sensitive protein to the acoustical energy. The sensory unit may have been genetically modified to express the acoustically sensitive transmembrane protein. The acoustically sensitive transmembrane protein may be selected from the group consisting of: PIEZO1, PIEZO2, MscMJ, MscS, MscL, MEC4, TRPY, TREK-1, TRP1, TRP4, TREK-1, TREK-2, Nav1.5, and TRAAK. The acoustical source may be selected from the group consisting of: a piezoelectric transducer, a composite transducer, a micromachined ultrasound transducer, a capacitive micromachined ultrasonic transducer, and a micro-electro-mechanical system. The acoustical source may comprise a silicon-on-insulator type micro-electro-mechanical system.

### SUMMARY

One embodiment is directed to an implantable probe system for delivering acoustical energy to a targeted tissue portion of a patient, comprising: a plurality of substrate portions, each substrate portion comprising at least one acoustical emitter; a probe body portion having proximal and distal ends and being movably coupled to the plurality of substrates and configured to at least partially encapsulate the plurality of substrates; and a distal end portion coupled to the distal end of the probe body portion, the distal end portion comprising at least one guiding feature configured to redirect a path of at least one of the substrate portions as such substrate portion is extended through and past the distal end portion by moving the plurality of substrates relative to the probe body portion. The implantable probe system further may comprise an ejector portion configured to move the plurality of substrates relative to the probe body portion. The ejector portion may comprise an elongate member configured to advance the plurality of substrates relative to the probe body portion, wherein the elongate portion is coupled to the plurality of substrates. The implantable probe system further may comprise a power source operatively coupled to the at least one acoustical emitter and configured to provide power to activate the at least one acoustical emitter. The elongate member may comprise a structure selected from the group consisting of: a wire, a fiber, a rod, and a tube. The elongate member may comprise a material selected from the group consisting of: a polymer, and a metal. The implantable probe system further may comprise a collar member, the collar member coupled to both the elongate member and the plurality of substrates. The targeted tissue portion may be selected to be an acoustically sensitive tissue portion. The targeted tissue portion may comprise one that has been configured to express an acoustically sensitive transmembrane protein. The targeted tissue portion may be one that has been genetically modified to express the acoustically sensitive transmembrane protein. The acoustically sensitive transmembrane protein may be selected from the group consisting of: PIEZO1, PIEZO2, MscMJ, MscS, MscL, MEC4, TRPY, TREK-1, TRP1, TRP4, TREK-1, TREK-2, Nav1.5, and TRAAK. At least one of the plurality of substrates may comprise a plurality of acoustical emitters. The plurality of acoustical emitters may be configured to direct energy in at least two different directions.

Another embodiment is directed to a system for altering the function of a sensory unit that innervates a targeted tissue portion of an animal, the system comprising an acoustical source configured to be operatively coupled to an exposed surface of the animal and to provide acoustical energy to the targeted tissue portion, wherein the sensory unit has been configured to express an acoustically sensitive transmembrane protein, such that when the targeted tissue portion is exposed to acoustical energy transcutaneously from the acoustical source, a membrane potential of cells comprising the targeted tissue structure is modulated at least in part due to exposure of the acoustically sensitive protein to the acoustical energy. The sensory unit may have been genetically modified to express the acoustically sensitive transmembrane protein. The acoustically sensitive transmembrane protein may be selected from the group consisting of: PIEZO1, PIEZO2, MscMJ, MscS, MscL, MEC4, TRPY, TREK-1, TRP1, TRP4, TREK-1, TREK-2, Nav1.5, and TRAAK. The acoustical source may be selected from the group consisting of: a piezoelectric transducer, a composite transducer, a micromachined ultrasound transducer, a capacitive micromachined ultrasonic transducer, and a micro-electro-mechanical system. The acoustical source may comprise a silicon-on-insulator type micro-electro-mechanical system.

### BRIEF DESCRIPTION OF THE DRAWINGS

FIG. 1 illustrates one embodiment of a configuration for an acoustics-based neuromodulation therapy.

FIG. 2 illustrates an embodiment of system level componentry configuration for treatment of a human in accordance with the present invention.

FIG. 3 illustrates an embodiment of system level componentry configuration for treatment of a human in accordance with the present invention.

FIG. 4 illustrates an embodiment of system level componentry configuration for treatment of a human in accordance with the present invention.

FIG. 5 illustrates one embodiment of a probe configuration.

FIG. 6 illustrates an embodiment of a probe configuration.

FIG. 7 illustrates an embodiment of a probe configuration.

FIG. 8 illustrates an embodiment of a probe configuration.

FIG. 9 illustrates an embodiment of a probe configuration.

FIG. 10 illustrates an embodiment of a probe configuration.

FIG. 11 illustrates an embodiment of a probe configuration.

FIG. 12 illustrates an embodiment of a probe configuration.

FIG. 13 illustrates an embodiment of a probe configuration.

FIG. 14 illustrates an embodiment of a probe configuration.

FIG. 15 illustrates an embodiment of a probe configuration.

### DETAILED DESCRIPTION

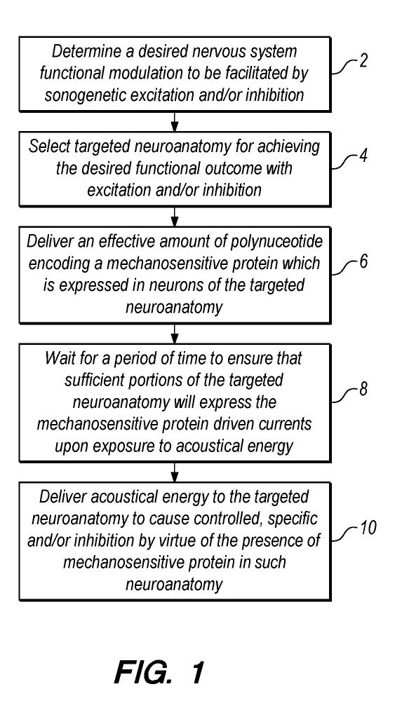

Referring to FIG. 1, from a high-level perspective, a songenetics-based neuromodulation intervention involves determination of a desired nervous system functional modulation which can be facilitated by sonogenetic excitation and/or inhibition (2), followed by a selection of neuroanatomic resource within the patient to provide such outcome (4), delivery of an effective amount of polynucleotide encoding a mechanoresponsive protein which is expressed in neurons of the targeted neuroanatomy (6), waiting for a period of time to ensure that sufficient portions of the targeted neuroanatomy will indeed express the mechanoresponsive protein-driven currents upon exposure to acoustical energy (8), and delivering acoustical energy to the targeted neuroanatomy to cause controlled, specific excitation and/or inhibition of such neuroanatomy by virtue of the presence of the mechanoresponsive protein therein (10) that may modulate the membrane potential of a neuron, or other cell by transporting ions through the membrane.

Intensity threshold ranges of 3×10⁻⁴-1.25 W/cm² are routinely used at frequencies of 1-20 MHz for diagnostic applications that produce pressures of 0.5-6 MPa. Intensities of 200-10,000 W/cm² are routinely used at frequencies of 0.2-10 MHz for ablative therapies that produce pressures of 5-30 MPa.

As used in existing ablative therapies, a series of sonications with powers typically ranging between 150 and 250 W may be used to produce temperatures increases of between 3 and 8° C. to confirm accurate focusing using magnetic resonance thermography (MRT). Subsequently, therapeutic sonications typically ranging from 10 to 20 s may be implemented by gradually escalating the power and monitoring until a maximum MRT voxel temperature increase reaches between 13 and 26° C.

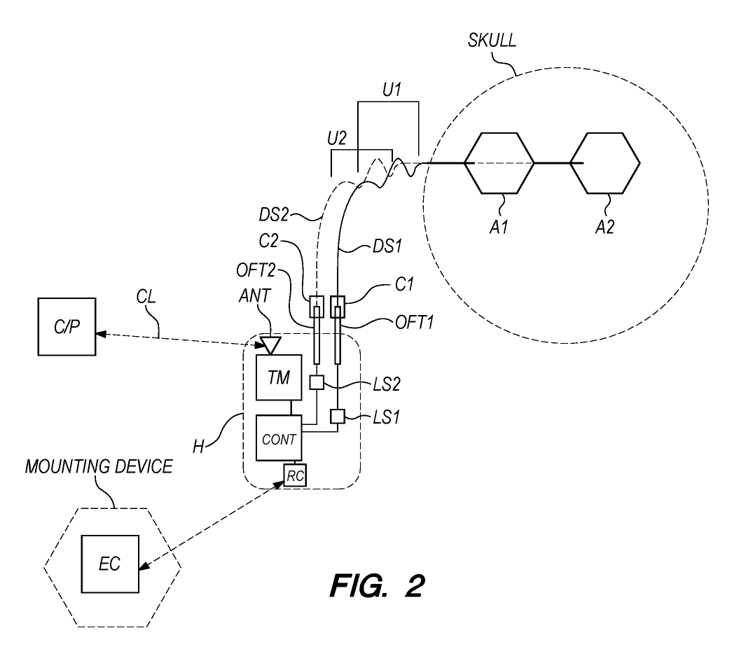

In certain embodiments of the present invention, an intensity range of 0.05-10 W/cm² may be utilized at frequencies of 20-1000 kHz to produce a peak negative pressure of between 0.05 and 3 MPa within a target in the brain of patient. Such a system is shown below in FIG. 2.

Referring to FIG. 2, an embodiment of the present invention is illustrated that is configured for transcranial treatment of the brain of a patient. It should be noted that transcranial delivery also includes transcutaneous delivery. Applicators A1 & A2, may be deployed onto the skull to address a treatment target within the brain if a patient. Electrical energy is delivered to Applicators A1 & A2 via Delivery Segments DS1 & DS2, respectively. Although not shown for simplicity and clarity in the present figure, multiple applicators and/or delivery segments may be used for a specific target structure if it is a large target structure when compared to the acoustical penetration depth within that structure, or to create a phased array configuration. Delivery Segments DS1 & DS2 may be configured to be Pt/Ir alloy wires contained within 300 μm OD silicone or PEEK tubes whose distal end may be sealed with a biocompatible material, such as but not limited to epoxy. Connectors C1 & C2 are configured to operatively couple energy from Delivery Segments DS1 & DS2 to Applicators A1 & A2, respectively, and provide system modularity for ease of repair and/or replacement. Delivery Segments DS1 & DS2 may further comprise Undulations U1 & U2, respectively, which may provide strain relief. Delivery Segments DS1 & DS2 are operatively coupled to Housing H via Feedthroughs OFT1 & OFT2, respectively. Energy is provided to Delivery Segments DS1 & DS2 from Sources LS1 & LS2, respectively, within Housing H.

Devices suitable for practicing the present invention may comprise one or more components for generating acoustic or ultrasound waves, such as acoustic and ultrasonic emitters, transducers or piezoelectric transducers, composite transducers, micromachined ultrasound transducers (MUTs) including capacitive micromachined ultrasonic transducers (cMUTs), Micro-Electro-Mechanical Systems (MEMS), silicon on insulator MEMS (SOI MEMS). A device for generating ultrasound waves may be provided as single or multiple transducers or in array configurations. The ultrasound waves may be of any shape, and may be focused or unfocused. Focal spot size depends on probe active aperture diameter (A), wavelength ( [symbol] lambda) and focal length (F). The center deflection of a clamped circular plate under a uniform pressure can be found from the following equation for a circular membrane

$$P = \frac{{Eh}^{4}}{{R}^{3}} [ \frac{16 y}{3 ( 1 - {v}^{2} ) h} + \frac{( 7 - v ) {y}^{3}}{3 ( 1 - {v}^{2} ) {h}^{3}} + \frac{4 {R}^{2} σ y}{( 1 - v ) {Eh}^{3}} ]$$

where P is the uniform pressure applied on the membrane, R is the membrane radius, y is the center deflection, [symbol] is the intrinsic stress of the membrane material, E is the Young's modulus of the membrane material, and [symbol] is Poisson's ratio of the membrane material. This equation can be used to estimate the pressure of the ultrasound waves under a prescribed membrane deflection. Such emitters may be made atop a substrate. Multiple substrates may be combined to form a single applicator. Multiple applicators may be combined to form a single probe.

A suitable mechanoreceptor may be selected from the group containing; PIEZO1, PIEZO2, MscMJ, MscS, MscL, MEC4, TRPY, TREK-1, TRP1, TRP4, TREK-1, TREK-2, Nav1.5, and TRAAK, but others are considered within the scope of the present invention.

There are a number of potential methodologies that can be used to deliver the mechanoreceptor to cells in vivo. These include viral mediated gene delivery, electroporation, optoporation, ultrasound, hydrodynamic delivery, or the introduction of naked DNA either by direct injection or complemented by additional facilitators such as cationic lipids or polymers. For example, the blood-brain barrier (BBB) may be made more permeable by generating pressures of about 0.3 MPa @ 260 kHz using localized average powers of about 10 mW with a 10% duty cycle on a PRF of 1 Hz without immediate collateral damage. Further safety margin may be achieved by reducing the duty cycle of the source to commensurately reduce the average power. It has been observed experimentally that the BBB permeability may remain improved to 50% effect for as long 60 s after sonication.

Viral expression systems have the dual advantages of fast and versatile implementation combined with high copy number for robust expression levels in targeted neuroanatomy. Cellular specificity may be obtained with viruses by virtue of promoter selection if the promoters are small and specific, by localized targeting, and by restriction of mechanoreceptor activation (i.e., via targeted sonication) of particular cells or projections of cells. In one embodiment, a mechanoreceptor is targeted by methods described in Yizhar et al. 2011, Neuron 71:9-34, which is incorporated by reference herein in its entirety. In addition, different serotypes of the virus (conferred by the viral capsid or coat proteins) will show different tissue tropism.

Lenti- and adeno-associated viral (“AAV”) vectors have been utilized successfully to introduce exogenous proteins into the mouse, rat and primate brain. Other vectors include but are not limited to equine infectious anemia virus pseudotyped with a retrograde transport protein (e.g., Rabies G protein), and herpes simplex virus (“HSV”). Additionally, these have been well tolerated and highly expressed over relatively long periods of time with no reported adverse effects, providing the opportunity for long-term treatment paradigms. Lentivirus, for example, is easily produced using standard tissue culture and ultracentrifuge techniques, while AAV may be reliably produced either by individual laboratories or through core viral facilities.

AAV is a preferred vector due to its safety profile, and AAV serotypes 1 and 6 have been shown to infect motor neurons following intramuscular injection in primates. Additionally, AAV serotype 2 has been shown to be expressed and well tolerated in human patients.

AAV6 may be a preferred serotype for intraneural injections as it has been demonstrated to preferentially infect nociceptive fibers following nerve injection in rodents.

AAV8 may be a preferred serotype for intrathecal injections as it has been demonstrated to efficiently transduce DRG neurons following lumbar puncture in rodents, dogs and pigs.

AAV5 may be a preferred serotype for direct DRG injections as it has high neural tropism when injected into rodent and primate brains, but also, has low tropism for axons of passage, which may be important to restrict expression from motor neurons which have axons of passage adjacent to the DRG. AAV2 may also be a preferred serotype for direct DRG injections as it has experience in neural parenchyma injections in the humans, and also, has limited tropism for axons of passage.

Viral expression techniques, generally comprising delivery of DNA encoding a desired mechanoreceptor and promoter/catalyst sequence packaged within a recombinant viral vector may be utilized to effectively transfect targeted neuroanatomy and deliver genetic material to the nuclei of targeted neurons, thereby inducing such neurons to produce mechanoreceptor proteins which are migrated throughout the neuron cell membranes where they are made functionally available to sonication components of the interventional system. Typically, a viral vector will package what may be referred to as a “mechanoreceptor expression cassette”, which will contain the mechanoreceptor (e.g., PIEZO2, TRP4, etc.) and a promoter that will be selected to drive expression of the particular mechanoreceptor within a targeted set of cells. In the case of adeno-associated virus (or AAV), the gene of interest (mechanoreceptor) can be in a single stranded configuration with only one mechanoreceptor expression cassette or in a self-complementary structure with two copies of mechanoreceptor expression cassette complimentary in sequence with one another and connected by hairpin loops. The self-complementary AAVs are thought to be more stable and show higher expression levels and shows faster expression. The promoter may confer specificity to a targeted tissue, such as in the case of the human synapsin promoter (“hSyn”) or the human Thy1 promoter (“hThy1”) which allow protein expression of the gene under its control in neurons. Another example is the calcium/calmodulin-dependent kinase II promoter (“CAMKII”), which allows protein expression of the gene under its control only in excitatory neurons, a subset of the neuron population. Alternatively, a ubiquitous promoter may be utilized, such as the human cytomegalovirus (“CMV”) promoter, or the chicken beta-actin (“CBA”) promoter, each of which is not particularly neural specific, and each of which has been utilized safely in gene therapy trials for neurodegenerative disease. Viral constructs carrying mechanoreceptor may be optimized for specific neuronal populations and are not limited to such illustrative examples.

In one embodiment, nerve fibers may be targeted by direct injection (i.e., injection into the nerve itself). This approach, which may be termed “intrafascicular” or “intraneural” injection, involves placing a needle into the fascicle of a nerve bundle. Intrafascicular injections are an attractive approach because they allow specific targeting those neurons which may innervate a relatively large target (e.g., fibers across entire kidney, fibers across entire dermatome of skin, fibers across entire stomach wall) with one injection (e.g., before the fibers enter the tissue and anatomically bifurcate). The pertinent vector solution may be injected through the needle where it may diffuse throughout the entire nerve bundle (10 to thousands of axon fibers). The vector may then enter the individual axon fibers through active (receptor-mediated) or passive (diffusion across intact membranes or transiently disrupted membranes) means. Once it has entered the axon, the vector may be delivered to the cell body via retrograde transport mechanisms, as described above. The number of injections and dose of virus injected to the nerve are dependent upon the size of the nerve, and can be extrapolated from successful transduction studies. For example, injection of the sciatic nerve of mice (approximately 0.3 mm diameter) with 0.002 mL saline containing 1×10⁹ vg of AAV has been shown to result in efficient transgene delivery to sensory neurons involved in pain sensing. Likewise, injection of the sciatic nerve of rats (1 mm diameter) with 0.010 mL saline containing 1-4×10¹⁰ vg of AAV has also achieved desirable transfection results. The trigeminal nerve in humans is 2 mm in diameter, and through extrapolation of the data from these pertinent studies, the trigeminal nerve may be transfected to efficiently deliver a transgene to these pertinent pain neurons using a direct injection of 0.05 mL saline containing 4×10¹⁰×10¹⁴ vg of AAV into the trigeminal bundle. These titers and injection volumes are illustrative examples and are specifically determined for each viral construct-target neuron pairing.

As mentioned above, injection into the ganglion may be utilized to target the neural cell bodies of peripheral nerves. Ganglia consist of sensory neurons of the peripheral nervous system, as well as autonomic neurons of the parasympathetic and sympathetic nervous system. A needle may be inserted into the ganglion which contains the cell bodies and a vector solution injected through the needle, where it may diffuse throughout the tissue and be taken up by the cell bodies (hundreds, to thousands, of cells). In one embodiment, a dose of approximately 0.1 mL saline containing from 1×10¹¹ vg to 1×10¹⁴ vg of AAV may be used per ganglion. There are different types of ganglia that may be targeted. Dorsal root ganglion of the spinal cord may be injected in a similar method that is used during selective dorsal rhizotomy (i.e. injection via the intrathecal subarachnoid space of the spinal cord), except rather than cutting the nerves, the dorsal root ganglia may be injected. Other ganglia not in the abdominal cavity, such as the nodose ganglion of the vagus nerve, may be targeted by making an incision through the skin, and then exposing the ganglia through separation of muscles, fascia and tendons. Ganglia in the abdominal cavity, such as the ganglia of the renal plexus, may be injected through laparoscopic techniques, wherein one or more small incisions may be made through the skin and abdominal wall to allow insertion of the surgical apparatus (camera, needle, tools, etc.) to locations facilitating access and imaging of the pertinent targeted tissue. The needle may be guided into the ganglia (as visualized through a camera or other imaging device, such as ultrasound or fluoroscopy). In all cases, the vector solution may be injected as a single bolus dose, or slowly through an infusion pump (0.001 to 0.1 mL/min). These ranges are illustrative, and doses are tested for each virus-promoter-mechanoreceptor construct pairing them with the targeted neurons.

In one particular example of ganglion injection, the dorsal root ganglia mediating clinical neuropathic pain may be injected with an AAV vector solution, preferably containing an AAV vector that has tropism for cell body.

In another particular example of ganglion injection, the dorsal root ganglia mediating undesired muscular spasticity may be injected with an AAV vector solution. An AAV vector that has tropism for cell body may be used towards this goal, as is described elsewhere herein.

Spatial specificity be attained via the biological specificity of the vector, as mentioned above, or via localized infusion of the gene therapy agent. Therefore, focusing of the excitation energy is not necessarily required for sonogenetic treatment specificity. However, it may be beneficial when using mechanosensitive channels with higher activation thresholds to avoid collateral damage to intermediate tissue.

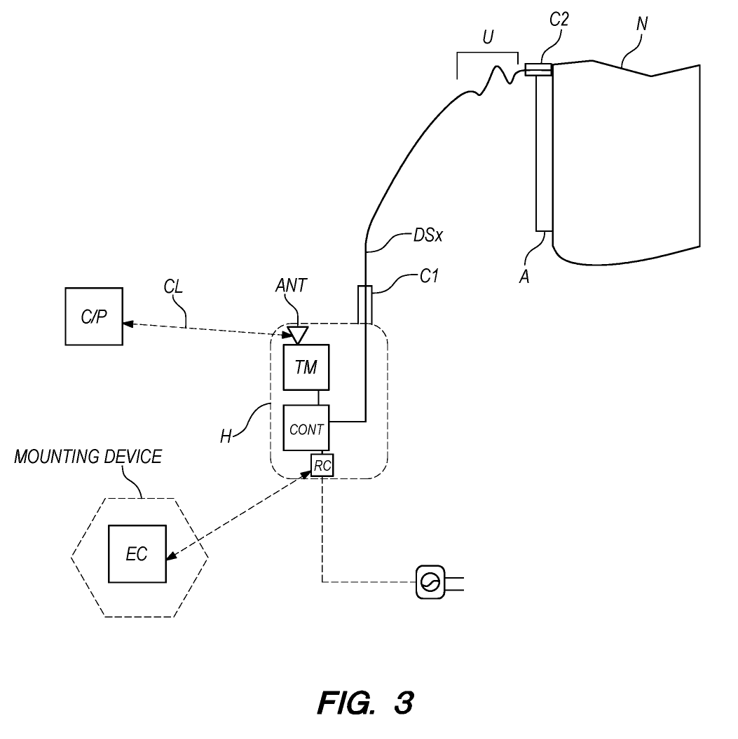

FIG. 3 relates to an embodiment of the present invention similar to that of FIG. 2 with the differences of being further configured to address targets in the peripheral nervous system and to deliver energy trancutaneously. Applicator A may be a slab-type applicator that is 20 mm wide and 40 mm long which is deployed about the surface of target tissue N. Electrical power may be delivered to Applicator A via Delivery Segment DS to power the transducer(s) resident in the applicator. The system may be operated in a pulsed burst mode, where the burst duration may be made from between 0.5 ms to 1 s. Furthermore, the pulse repetition frequency (PRF) may be configured from between 0.1 Hz and 200 Hz, with a PRF of 1 Hz being typically effective. Consequently, the duty cycle ranges from 0.005% to 100%, with a duty cycle of 1% being typically effective. Although not shown for simplicity and clarity in the present figure, multiple applicators and/or delivery segments may be used for a specific target structure if it is a large target structure when compared to the acoustical penetration depth within that structure. Delivery Segment DS may be configured to be a ribbon cable. Delivery Segment DS may be configured to be a monolithically formed silicon ribbon cable. Delivery Segment DS may further comprise Undulations U, which may provide strain relief. Delivery Segment DS may be operatively coupled to Housing H via connector C1 and to the applicator via connector C2. The electrical power and/or current may be controlled by controller CONT, and parameters such as acoustical intensity, exposure time, pulse duration, pulse repetition frequency, and duty cycle may be configured. The Controller CONT shown within Housing H is a simplification, for clarity, of that described in more detail with respect to FIG. 10. External clinician programmer module and/or a patient programmer module C/P may communicate with Controller CONT via Telemetry module TM via Antenna ANT via Communications Link CL. Power Supply PS, not shown for clarity, may be wirelessly recharged using External Charger EC. Furthermore, External Charger EC may be configured to reside within a Mounting Device MOUNTING DEVICE. Mounting Device MOUNTING DEVICE may be a vest, as is especially well configured for this exemplary embodiment. External Charger EC, as well as External clinician programmer module and/or a patient programmer module C/P and Mounting Device MOUNTING DEVICE may be located within the extracorporeal space ESP, while the rest of the system is implanted and may be located within the intracorporeal space ISP. External Charger EC may also be an AC adapter, as shown by the dotted line and universal AC symbol. Various other embodiments of applicators A and other system components, configurations, and methods are described, for example, in U.S. patent application Ser. Nos. 14/737,445 and 14/737,446, both entitled, “Optogenetic Therapies for Movement Disorders”, which are incorporated by reference herein in their entireties.

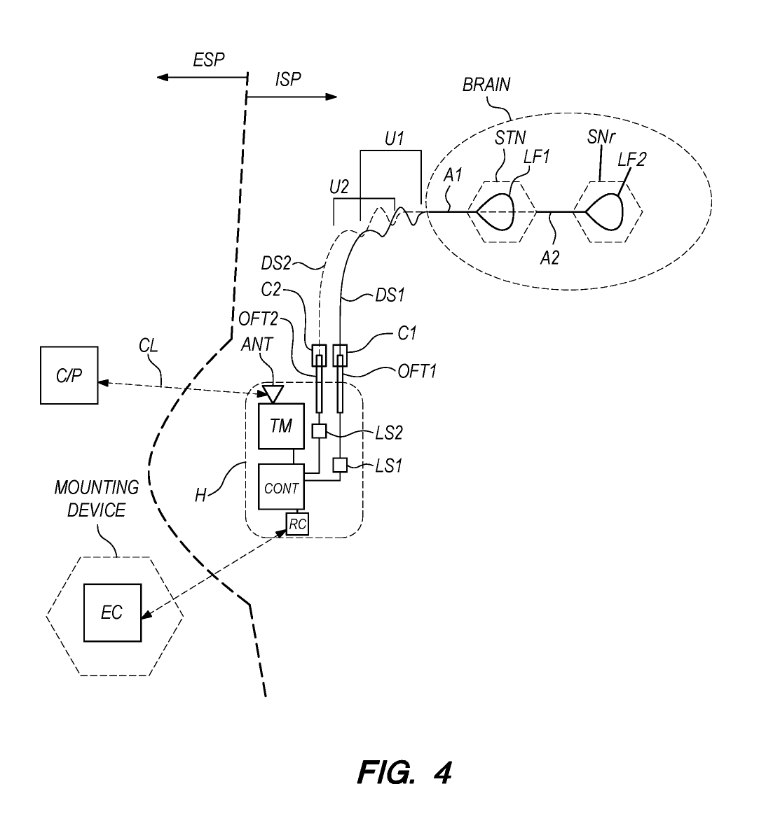

FIG. 4 relates to an embodiment of the present invention similar to that of earlier embodiments further configured to utilize an implanted sonicator (a.k.a an “acoustical emitter” or “acoustical source”) within the target tissue. Applicators A1 & A2, may be deployed within the Brain itself, which contains the STN and the SNr in this example directed at the treatment of Parkinson's Disease. Electrical energy is delivered to Applicators A1 & A2 via Delivery Segments DS1 & DS2, respectively, to create Acoustical Fields LF1 & LF2, respectively, within the target tissues. Acoustical Fields LF1 & LF2 may be configured to provide sonication of the target tissues within the intensity range of 0.01-1000 mW/mm² to provide for a reasonable volume of tissue within which the intensity is at or above the activation threshold, and may be dependent upon one or more of the following factors; the specific mechanoreceptor used, its concentration distribution within the tissue, the tissue acoustical properties, and the size of the target structure(s). Although not shown for simplicity and clarity in the present figure, multiple applicators and/or delivery segments may be used for a specific target structure if it is a large target structure when compared to the acoustical penetration depth within that structure. Delivery Segments DS1 & DS2 may be configured to be Pt/Ir alloy wires contained within 300 μm OD silicone or PEEK tubes whose distal end may be sealed with a biocompatible material, such as but not limited to epoxy. Connectors C1 & C2 are configured to operatively couple energy from Delivery Segments DS1 & DS2 to Applicators A1 & A2, respectively. Delivery Segments DS1 & DS2 further comprise Undulations U1 & U2, respectively, which may provide strain relief. Delivery Segments DS1 & DS2 are operatively coupled to Housing H via Feedthroughs OFT1 & OFT2, respectively. Energy is provided to Delivery Segments DS1 & DS2 from Sources LS1 & LS2, respectively, within Housing H. Delivery segments may comprise an additional electrical conductor, such as a wire, or be a portion of the substrate comprising the acoustical source, or a combination of an additional electrical conductor and a substrate.

Alternately, the Applicators in the above embodiments may be replaced by the opto-acoustic systems and transducers, such as those described, for example, in U.S. Pat. No. 6,022,309 and U.S. Pat. Appl. No. 20050021013, which are incorporated by reference herein in their entirety.

Applicators may comprise a substrate and at least a single acoustical emitter.

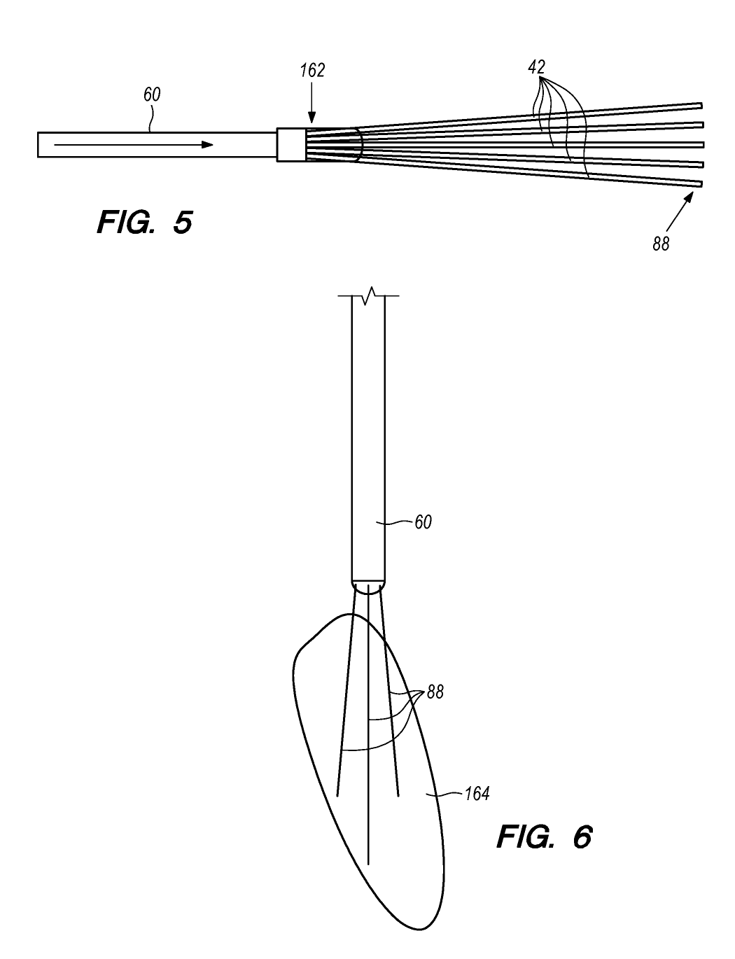

An emitter may be attached to the distal end of a substrate to form a probe. Probe body 60 may be configured to utilize diverter 162 (or “deflector”), such as that illustrated in FIG. 5, may be needed to maintain a lateral separation of the applicators 88. The diverter may also be used to deploy the substrate with the diffusers once the probe is placed in the tissue. The closer together the applicators, the greater the increase of the acoustical energy and/or power density between applicators by their mutual contributions may be. Likewise, the farther they are spread, the larger the volume may be. However, at too large of a distance the individual acoustical fields emitted by each diffuser may become separate and thereby leave gaps in the regions between applicators that fall below the activation threshold level of the acoustically sensitive protein that result in untreated portions of the target tissue.

Alternately, a probe may be constructed such that the applicators do not form a symmetrical pattern. Such a configuration may be useful, for example, when a target is itself asymmetrical, such as the STN, or when the target presents obliquely, or off-axis due to the surgical access route.

FIG. 6 illustrates an alternate embodiment configured for use with an asymmetrical target. In this exemplary embodiment, target 164 may be non-regular shape, as is often found in anatomy, and applicators 88 of probe body 60 may be configured to differing lengths, and/or diverter angles to accommodate the idiosyncrasies of the target. For example, the STN is generally biconvex-shaped structure that resembles a lens, or lenticule. Surgical access to the STN in humans may be made in a parasagittal plane moving rostral to caudal at an angle of 70° to the orbitomeatal line. In this configuration, the STN may present to the probe as an oblique oblate ellipsoid. Thus, the applicators may have a degree of symmetry, but not be completely symmetric (e.g. not radially symmetric about a center applicator).

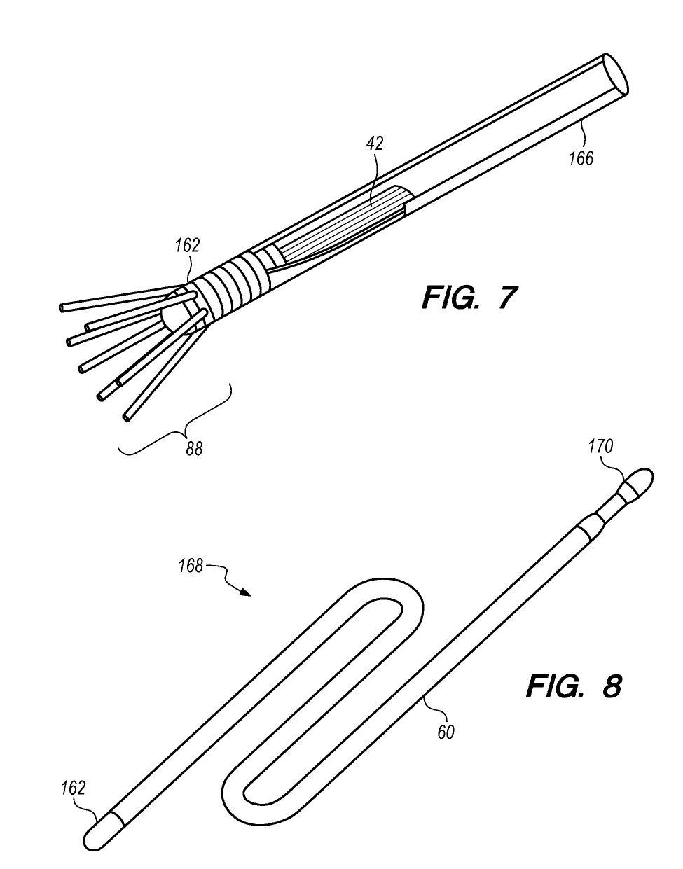

FIG. 7 illustrates a partially cut-away view of an embodiment of a probe body 60 further configured to utilize a diverter 162 to spread applicators 88 into a region of target tissue 164 (not shown) and provide an applicator separation distance, as described above. Such a configuration may provide the benefits of minimizing the size of the implant and an enhanced exposure volume. In this example, a plurality of substrates 42 that each feed an individual applicator 88 may be enclosed within a probe body that in turn may be covered with a biocompatible polymeric outer jacket 166 (such as polyurethane, for example) to prevent cellular ingrowth and contamination within the probe that may make its later removal more difficult.

FIG. 8 illustrates a further embodiment, where a probe 168 is displayed in its entirety, including a proximal connector 170 that may be used to couple the probe to a trunk cable to form a system, diverter housing 162 and a flexible probe body 60.

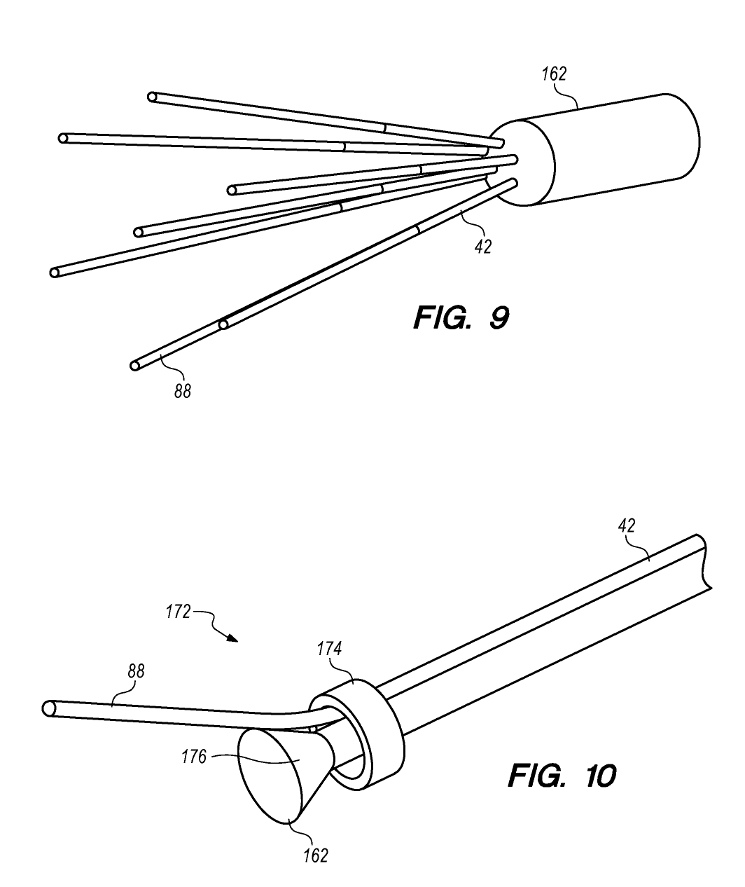

FIG. 9 illustrates a further embodiment, where more details regarding the deployment of applicators 88 are shown. Electrical energy may be conveyed to emitters within applicators 88 by substrates 42, all of which may be advanced through diverter housing 162 to create a pattern of applicators in target tissue. The substrates may be contained in a blunt-nosed, tubular probe that is similar in materials, size and flexibility to those used for deep brain stimulation (DBS). For consistency with earlier examples and embodiments, a radially symmetric 7-hexagonal configuration is shown in various figures herein, although other configurations and applicator/substrate numbers are considered within the scope of the present invention. The trajectory of the individual substrates may be defined by a diverter that directs the applicators as they are extended out of the probe tip (advanced) and into the target area.

Diagram 172 of FIG. 10 illustrates the basic concepts of a diverter, comprising a single substrate 42 terminating in an applicator 88, a diverter 162, a guide surface 176, and a containment ring 174 surrounding at least partially applicator 42 and guide surface 176. Guide surface 176 and containment ring 174 constrain substrate 42 and/or applicator 88 to deflect, or deviate from an otherwise nominally straight trajectory. That is to redirect the path of the substrate as it advances. It may be seen that a radially distributed plurality of such substrates in this exemplary configuration would nominally define a cone, as was shown in relation to the examples of FIGS. 5-9.

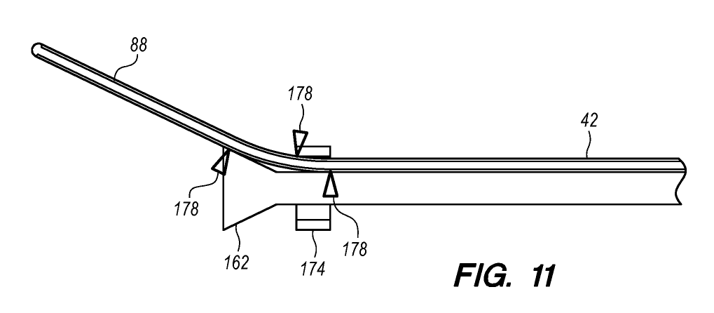

FIG. 11 expands upon the exemplary diverter shown in FIG. 10, with the added detail of contact points 178. Contact points 178 may define the deflection angle and radius of curvature of substrate 42. The contact points may also be surfaces, regions, or lines of contact to accomplish the same effect to form a guiding feature that deflects the applicator and/or substrate; which may form at least a partial channel to guide substrate 42 and/or emitter resident upon or within applicator 88.

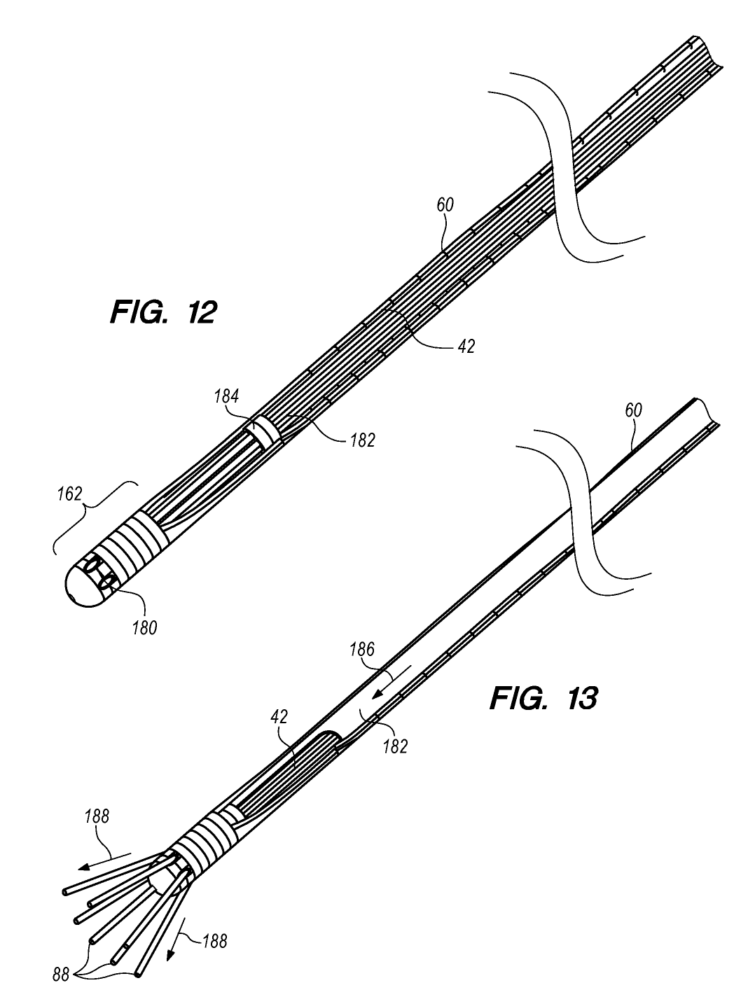

FIG. 12 illustrates an alternate embodiment of an integrated probe assembly 168, comprising probe body 60 with substrates 42 contained therein, a diverter tip 162 further comprising ports 180 therein, and collar 184 that is attached to the substrates and/or the applicators. Collar 184 may be advanced distally to push the applicators into the target tissue by means of ejector 182. Ejector 182 may be a push rod, a sheath inside of the probe body, for example. A probe body 60 may be comprised of a polymer tube, an elastomer tube, a metal tube, a coil/spring, or a combination of thereof, for example. By way of nonlimiting example, a metal tube may comprise a cut in its wall to increase its flexibility, like an interrupted helical cut, for example. By way of nonlimiting example, a probe body 60 with a cut in its outer surface may be further configured to comprise a coating or cover 166 to provide a barrier and thereby reduce the open area that may allow for ingress of fluids and infiltrates. By way of nonlimiting examples, an exterior sheath or covering 166 may be chosen from the list containing; a conformal coating, a polymer coating, an elastomer coating, a silicone coating, a parylene coating, and a hydrophobic coating. The diverter tip may, for example, be fabricated from metal, such as stainless steel, or a polymer, such as PTFE, using screw machining techniques.

FIG. 13 illustrates an alternate embodiment of the probe of FIG. 12, with the additions of the ejector being an inner sheath that has been advanced in direction 186 to advance applicators 88 in deployment direction 188, as indicated by arrows.

Alternately, the applicators may be configured in pre-set bends that serve, at least partially, to create separation between applicators. For example, a tube may be used to contain the applicator and/or substrate(s), with the tube further comprising a thermally induced shape set that may be made by heating the tube and/or the tube/applicator assembly to a temperature sufficient for plastic deformation. The shape-set applicator may be then positioned inside the probe body through a diverter tip, or at least a port and deployed, say using an ejector, as described elsewhere herein. By way of nonlimiting examples, the tube for shape-setting may be selected from the group consisting of; PEEK, Polyurethane, Tecothane, and PVDF. The shape may be set into the tube by placing it in a preconfigured channel that is cut into a block, or a pair of matched blocks that is/are then controllably heated to a temperature that renders the plastic pliable, nominally <the material glass temperature, then cooling it in place to set the shape. For example, PEEK 381G tubing may be placed in a pair of mating aluminum blocks, each containing a channel that consists of a straight section and curved section comprising 20° of a 10 mm radius of curvature. The channel may be cut using a ball endmill that is nominally only slightly larger than the outer diameter of the tubing. The blocks may be heated to from room temperature to a temperature of 85° C. over a period of 5-10 minutes and left to set for 30 seconds and then allowed to passively cool to room temperature in air over a period that is no shorter than 10 minutes to provide a uniform circular preset shape without undue residual stresses. Alternately, instead of polymeric compounds Nitonol may be used as sheath or as a guide onto which at least a single substrate may be affixed or adhered to provide a predefined shape to an applicator.

In a further alternate embodiment, a combination of pre-shaped applicators and a diverter may be used to provide nominally parallel applicator segments in tissue, as described above.

A constant curve, such as a circle or a line, may be preferable in order to avoid bisecting tissue during applicator deployment. In this manner, the applicator may follow a smooth, continuous path without lateral deviation.

Furthermore, a radio-opaque material may be used in the probe assembly to provide location information for intraoperative or postoperative imaging. Examples of radio-opaque materials are BaSO₄, metals and RO PEEK tubing, as is sold by Zeus, Inc., for use with applicators.

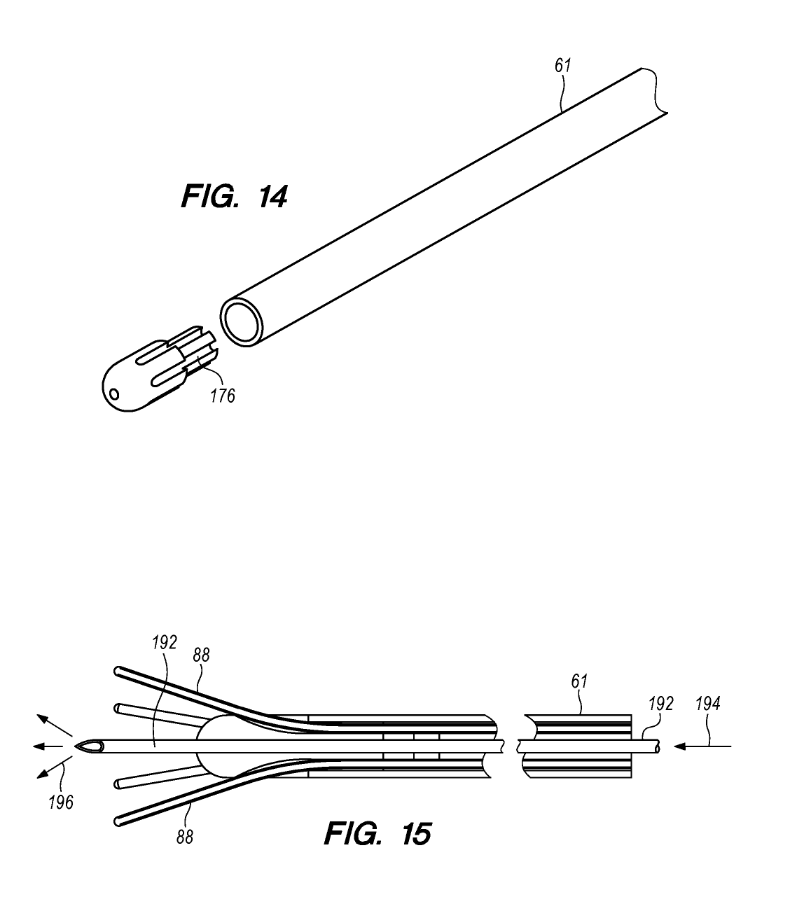

FIG. 14 illustrates an alternate embodiment, similar to that of FIGS. 12 and 13, wherein the diverter tip contains guide surfaces 176 and probe body 61 further comprises a subsumed containment ring 174 within the probe body itself.

FIG. 15 shows a further embodiment, with the addition of an infusion cannula 192 that may occupy, at least temporarily, a central lumen of probe 168. In this way, the same target tissue 164 may be accessed with a single probe insertion for both delivery of a gene therapy agent and, later for sonication. Arrows 196 indicate the direction of infusion of infusate 194.

As used herein, the terms “mechanosensitive”, “mechanically activated”, “mechanoreceptor”, “mechanotransduction”, “stretch-gated”, “acoustically sensitive”, and other similar terms of art are considered interchangeable.

Various exemplary embodiments of the invention are described herein. Reference is made to these examples in a nonlimiting sense. They are provided to illustrate more broadly applicable aspects of the invention. Various changes may be made to the invention described and equivalents may be substituted without departing from the true spirit and scope of the invention. In addition, many modifications may be made to adapt a particular situation, material, composition of matter, process, process act(s) or step(s) to the objective(s), spirit or scope of the present invention. Further, as will be appreciated by those with skill in the art that each of the individual variations described and illustrated herein has discrete components and features which may be readily separated from or combined with the features of any of the other several embodiments without departing from the scope or spirit of the present inventions. All such modifications are intended to be within the scope of claims associated with this disclosure.

Any of the devices described for carrying out the subject diagnostic or interventional procedures may be provided in packaged combination for use in executing such interventions. These supply “kits” may further include instructions for use and be packaged in sterile trays or containers as commonly employed for such purposes.

The invention includes methods that may be performed using the subject devices. The methods may comprise the act of providing such a suitable device. Such provision may be performed by the end user. In other words, the “providing” act merely requires the end user obtain, access, approach, position, set-up, activate, power-up or otherwise act to provide the requisite device in the subject method. Methods recited herein may be carried out in any order of the recited events which is logically possible, as well as in the recited order of events.

Exemplary aspects of the invention, together with details regarding material selection and manufacture have been set forth above. As for other details of the present invention, these may be appreciated in connection with the above-referenced patents and publications as well as generally known or appreciated by those with skill in the art. For example, one with skill in the art will appreciate that one or more lubricious coatings (e.g., hydrophilic polymers such as polyvinylpyrrolidone-based compositions, fluoropolymers such as tetrafluoroethylene, hydrophilic gel or silicones) may be used in connection with various portions of the devices, such as relatively large interfacial surfaces of movably coupled parts, if desired, for example, to facilitate low friction manipulation or advancement of such objects relative to other portions of the instrumentation or nearby tissue structures. The same may hold true with respect to method-based aspects of the invention in terms of additional acts as commonly or logically employed.

In addition, though the invention has been described in reference to several examples optionally incorporating various features, the invention is not to be limited to that which is described or indicated as contemplated with respect to each variation of the invention. Various changes may be made to the invention described and equivalents (whether recited herein or not included for the sake of some brevity) may be substituted without departing from the true spirit and scope of the invention. In addition, where a range of values is provided, it is understood that every intervening value, between the upper and lower limit of that range and any other stated or intervening value in that stated range, is encompassed within the invention.

Also, it is contemplated that any optional feature of the inventive variations described may be set forth and claimed independently, or in combination with any one or more of the features described herein. Reference to a singular item, includes the possibility that there are plural of the same items present. More specifically, as used herein and in claims associated hereto, the singular forms “a,” “an,” “said,” and “the” include plural referents unless the specifically stated otherwise. In other words, use of the articles allow for “at least one” of the subject item in the description above as well as claims associated with this disclosure. It is further noted that such claims may be drafted to exclude any optional element. As such, this statement is intended to serve as antecedent basis for use of such exclusive terminology as “solely,” “only” and the like in connection with the recitation of claim elements, or use of a “negative” limitation.

Without the use of such exclusive terminology, the term “comprising” in claims associated with this disclosure shall allow for the inclusion of any additional element—irrespective of whether a given number of elements are enumerated in such claims, or the addition of a feature could be regarded as transforming the nature of an element set forth in such claims. Except as specifically defined herein, all technical and scientific terms used herein are to be given as broad a commonly understood meaning as possible while maintaining claim validity.

The breadth of the present invention is not to be limited to the examples provided and/or the subject specification, but rather only by the scope of claim language associated with this disclosure.

What is claimed:

1. An implantable probe system for delivering acoustical energy to a targeted tissue portion of a patient, comprising:
A. a plurality of substrate portions, each substrate portion comprising at least one acoustical emitter;
B. a probe body portion having proximal and distal ends and being movably coupled to the plurality of substrates and configured to at least partially encapsulate the plurality of substrates; and
C. a distal end portion coupled to the distal end of the probe body portion, the distal end portion comprising at least one guiding feature configured to redirect a path of at least one of the substrate portions as such substrate portion is extended through and past the distal end portion by moving the plurality of substrates relative to the probe body portion.

2. The implantable probe system of claim 1, further comprising an ejector portion configured to move the plurality of substrates relative to the probe body portion.

3. The implantable probe system of claim 2, wherein the ejector portion comprises an elongate member configured to advance the plurality of substrates relative to the probe body portion, wherein the elongate portion is coupled to the plurality of substrates.

4. The implantable probe system of claim 1, further comprising a power source operatively coupled to the at least one acoustical emitter and configured to provide power to activate the at least one acoustical emitter.

5. The implantable probe system of claim 4, wherein the elongate member comprises a structure selected from the group consisting of: a wire, a fiber, a rod, and a tube.

6. The implantable probe system of claim 3, wherein the elongate member comprises a material selected from the group consisting of: a polymer, and a metal.

7. The implantable probe system of claim 3, further comprising a collar member, the collar member coupled to both the elongate member and the plurality of substrates.

8. The implantable probe system of claim 1, wherein the targeted tissue portion is selected to be an acoustically sensitive tissue portion.

9. The implantable probe system of claim 8, wherein the targeted tissue portion has been configured to express an acoustically sensitive transmembrane protein.

10. The implantable probe system of claim 9, wherein the targeted tissue portion has been genetically modified to express the acoustically sensitive transmembrane protein.

11. The implantable probe system of claim 9, wherein the acoustically sensitive transmembrane protein is selected from the group consisting of: PIEZO1, PIEZO2, MscMJ, MscS, MscL, MEC4, TRPY, TREK-1, TRP1, TRP4, TREK-1, TREK-2, Nav1.5, and TRAAK.

12. The implantable probe system of claim 1, wherein at least one of the plurality of substrates comprises a plurality of acoustical emitters.

13. The implantable probe system of claim 12, wherein the plurality of acoustical emitters is configured to direct energy in at least two different directions.

14. A system for altering the function of a sensory unit that innervates a targeted tissue portion of an animal, the system comprising an acoustical source configured to be operatively coupled to an exposed surface of the animal and to provide acoustical energy to the targeted tissue portion, wherein the sensory unit has been configured to express an acoustically sensitive transmembrane protein, such that when the targeted tissue portion is exposed to acoustical energy transcutaneously from the acoustical source, a membrane potential of cells comprising the targeted tissue structure is modulated at least in part due to exposure of the acoustically sensitive protein to the acoustical energy.

15. The system of claim 14, wherein the sensory unit has been genetically modified to express the acoustically sensitive transmembrane protein.

16. The system of claim 15, wherein the acoustically sensitive transmembrane protein is selected from the group consisting of: PIEZO1, PIEZO2, MscMJ, MscS, MscL, MEC4, TRPY, TREK-1, TRP1, TRP4, TREK-1, TREK-2, Nav1.5, and TRAAK.

17. The system of claim 14, wherein the acoustical source is selected from the group consisting of: a piezoelectric transducer, a composite transducer, a micromachined ultrasound transducer, a capacitive micromachined ultrasonic transducer, and a micro-electro-mechanical system.

18. The system of claim 17, wherein the acoustical source comprises a silicon-on-insulator type micro-electro-mechanical system.
